(19) United States
(12) Patent Application Publication
(10) Pub. No.: US 2021/0308095 A1
(43) Pub. Date: Oct. 7, 2021

(54) Cannabinoid-Containing Composition

(71) Applicant: Joyn Botanicals Ltd., Windsor (CA)

(72) Inventor: Joseph D. ARTISS, Windsor (CA)

(21) Appl. No.: 17/351,621

(22) Filed: Jun. 18, 2021

Related U.S. Application Data
(63) Continuation-in-part of application No. 16/225,964, filed on Dec. 19, 2018.
Application No. 16/225,964 is a continuation-in-part of application No. PCT/IB2019/060799, filed on Dec. 13, 2019.
Application No. PCT/IB2019/060799 is a continuation of application No. 16/225,964, filed on Dec. 19, 2018.

(51) Int. Cl. A61K 31/352; A61K 31/05; A61P 25/24; A61P 25/28; A23L 33/105; A23L 29/269; A61K 36/185

(52) U.S. Cl. CPC A61K 31/352; A61K 31/05; A61P 25/24; A61P 25/28; A23L 33/105; A23V 2002/00; A61K 36/185; A23V 2200/32; A23V 2250/5112; A23V 2250/2132; A23V 2200/31; A23L 29/273

(57) ABSTRACT

The invention relates to compositions comprising complexes of aqueous and non-aqueous α-cyclodextrin and cannabinoids and/or complexes of aqueous and non-aqueous α-cyclodextrin and terpenes, methods of making these α-cyclodextrin and cannabinoid complex-containing compositions, and methods of using the α-cyclodextrin and cannabinoid complex-containing compositions. The invention also relates to food products comprising the α-cyclodextrin and cannabinoid complex-containing compositions of this invention.

### CROSS-REFERENCE TO RELATED APPLICATIONS

The present application is a continuation-in-part of U.S. application Ser. No. 16/225,964, filed Dec. 19, 2018. The present application also claims the benefit of PCT International Application No. PCT/IB2019/060799, filed Dec. 13, 2019, which claims the benefit of U.S. application Ser. No. 16/225,964, filed Dec. 19, 2018.

The patent or application file contains at least one drawing executed in color. Copies of this patent or patent application publication with color drawings will be provided by the Office upon request and payment of the necessary fee.

### BACKGROUND

Cannabis contains more than 500 compounds, including at least 66 cannabinoids that are unique to Cannabis and at least 140 terpenes that are widespread throughout the plant kingdom. (Brenneisen, R., Chemistry and Analysis of Phytocannabinoids and Other Cannabis Constituents, in Forensic Science and Medicine: Marijuana and the Cannabinoids. Ed. El Sohly, M A. Humana Press Inc., Totowa, N.J. Chapter 2, pgs. 17-49. 2010.)

The term “phytocannabinoid” has been proposed to differentiate naturally-occurring cannabinoids from synthetically produced cannabinoids. Synthetically produced cannabinoids are cannabinoid compounds that are made synthetically in laboratories. Phytocannabinoids are all C₂₁ terpenophenolic compounds, of which tetrahydrocannabinol (THC) and cannabidiol (CBD) are perhaps the most studied and best understood for their therapeutic and recreational value. However, phytocannabinoids as a whole have become of interest for the treatment of various disorders and disease states, such as anxiety disorders, depression, loss of appetite (including cachexia), various forms of pain (e.g., acute pain, chronic pain, neuropathic pain, trigeminal nerve pain, and pain associated with or accompanying migraine headaches and cancer).

The 140 terpenes that are present in Cannabis include monoterpenoids, sesquiterpenoids, diterpenoids, and triterpenoids. (Brenneisen, pgs. 17-49.) For the most part, these terpenes are hydrophobic and comprise short aliphatic branched chains or substituted monocyclic compounds. Terpenes present in Cannabis are responsible for the unpleasant turpentine or pine needle tastes and odors that are common to Cannabis plant extracts.

Naturally-occurring cyclodextrins are cyclic polymers of glucose units that are formed by the enzymatic action of specific cyclodextrin glycosyltransferases (CGT) on partially-hydrolyzed corn starch. Depending on the CGT used, cyclic polymers of six, seven, or eight glucose units are produced, which are respectively named α-cyclodextrin, β-cyclodextrin, and γ-cyclodextrin. The size of the cyclodextrin molecule, and therefore the “pore” formed by the cyclodextrin molecule, is dictated by the number of glucose units in the polymer. Thus, α-cyclodextrin has the smallest pore, while γ-cyclodextrin has the largest. Alpha-cyclodextrin is known to sequester small molecules (e.g., fatty acids, nitrobenzene) (McGowan, M. et al., A peroxidase-coupled method for the colorimetric determination of serum triglycerides. Clin. Chem. 1983, 29, 538-542.), while β-cyclodextrin and γ-cyclodextrin sequester larger poly- and heterocyclic compounds (e.g., cholesterol, THC or CBD).

Shoyama describes a method for the preparation of pure THC and β-cyclodextrin complexes and demonstrates that these complexes are stable. (Shoyama Y. et al., Preparation and stability of Δ⁹-tetrathydrocannabinol-β-cyclodextrin inclusion complex, J. Nat. Prod. 1983, 46(5), 633-637.) Hazekamp assesses the water solubility of pure THC in the presence of α-cyclodextrin, β-cyclodextrin, γ-cyclodextrin, and randomly methylated-β-cyclodextrins and states that only the randomly methylated-β-cyclodextrins increase the water solubility of THC. (Hazekamp, A. et al., Structure elucidation of the tetrahydrocannabinol complex with randomly methylated β-cyclodextrin, Eur. J. Pharm. Sci. 2006, 29, 340-347.) Mannila et al. describe the preparation of β-cyclodextrin complexes with CBD for the sublingual administration of CBD and the preparation of methylated-cyclodextrin complexes with CBD and THC. (U.S. Pat. No. 7,592,328; Mannila J. et al., Precipitation complexation method produces cannabidiol/β-cyclodextrin inclusion complex suitable for sublingual administration of cannabidiol, J. Pharm. Sci. 2007, 96(2), 312-319.) U.S. Pat. No. 7,423,026 describes methylated β-cyclodextrin complexes with cannabinol, THC, and CBD. U.S. Pat. No. 8,735,374 describes the preparation of an oral mucoadhesive dosage form of THC and γ-cyclodextrin and a mixture of THC and CBD with a mixture that includes both β- and γ-cyclodextrin.

U.S. Patent No. 2017/0224842 (“Czap”) describes γ-cyclodextrin complexes of cannabinoids from partially purified, low terpene containing, hemp oil. In order to release the guest cannabinoid molecule from the host cyclodextrin and to alleviate the concerns surrounding undigested cyclodextrins, Czap's complexes are formulated with amylase-like enzymes that hydrolyze the cyclodextrin. Czap further discloses that a mixture of cyclodextrins may be used to sequentially form inclusion complexes with different size molecules. Czap's formulation must be kept dry to prevent premature activation of the amylase-like enzymes. Thus, Czap's formulation is not feasible for use in beverages, food stuffs, and confections. Furthermore, hot beverages or a baking process will likely denature the amylase-like enzymes in Czap's complexes.

WO 2017/180954 describes the emulsification of specific cannabinoids with a mixture of at least two emulsifiers, including a synthetic β-cyclodextrin. WO 2018/058235 describes the preparation of a complex using Cannabis plant extract, β-cyclodextrin, and other ingredients (e.g., release-modifying agents and excipients for use in a time-released tablet).

Shimada et al. (Shimada K, Kimiko K, Oshii J and Nakamura T. Structure of inclusion complexes of cyclodextrins with triglyceride at vegetable oil/water interface. J. of Food Science, 1992, 57 (3):655-656.), Artiss and Jen (Compositions comprising dietary fat complexer and methods for their use. US Pat. No. 6,890,549, May 10, 2005), Bochot et al. (Bochot A, Trichard L, Le Bas G, Alphandary H, Grossiord J L, Duchêne D and Fattal E. Internation α-Cyclodextrin/oil beads: An innovative self-assembling system. J of Pharmaceutics, 2007, 339:121-129.), Trichard et al. (Trichard L, Delgado-Charro M B, Guy R H, Fattal E and Bochot A. Novel beads made of alpha-cyclodextrin and oil for topical delivery of a lipophilic drug. Pharmaceutical Research, 2007, 25 (2):435-440.), and Bhopate SB and Dhole SN (Preparation and characterisation of β-cyclodextrin nebivolol inclusion complex. Int. J. of Pharmaceutical Sciences and Research, 2014, 6(5):2205-2213.) all describe the formation of cyclodextrin complexes with hydrophobic compounds in a water/oil mixture. For example, Bhopate and Dhhole describe five different methods for creating cyclodextrin complexes, each of which requires water as part of the solvent system. From these publications, we understand that an oil:water interface is required for exchange between the two phases. Furthermore, we learn that the hydrophobic target in the hydrophobic phase must complex with the water-soluble cyclodextrin in order for it to cross into the aqueous phase.

Wadhwa et al. (Wadha G, Kumar S, Chhabra L, Mahant S and Rao R. Essential oil-cyclodextrin complexes: an updated review. J Incl Phenom Macrocycl Chem, 2017, 89:39-58.), Al-Marzouqi et al. (Al-Marzouqi A H, Elwy H M, Shehadi I and Adem A. Physicochemical properties of antifungal drug-cyclodextrin complexes prepared by supercritical carbon dioxide and conventional techniques. J Pharm Biomed Analysis, 2009, 49:227-233.), and Cheirsilp and Rakmai (Cheirsilp B and Rakmai J. Inclusion complex formation of cyclodextrin with its guest and their applications, 2016, Biol Eng Med. 2(1):1-6.) discuss the preparation of inclusion complexes of cyclodextrins dissolved in water and guest molecules dissolved in an organic solvent.

Harada, Li and Kamachi (Harada A, Li J and Kamachi M. Preparation and properties of inclusion complexes of poly(ethylene glycol) with α-cyclodextrin. Macromolecules, 1993, 26:5698-5703.) and Bisson-Boutelliez et al. (Bisson-Boutelliez C, Fontanay S, Finance C and Kedzierewicz F. Preparation and physicochemical characterization of amoxicillin β-cyclodextrin complexes. AAPS Pharm Sci Tech., 2010, 11(2):574-580.) describe the formation of cyclodextrin guest inclusion complexes when both the cyclodextrin and the guest molecule are dissolved in water.

In their review article, Maazaoui and Abderrahim (Maazaoui R and Abderrahim R. Applications of cyclodextrins : formation of inclusion complexes and their characterization. Intern J Advan Res., 2015, 3(2):1030-1030.) describe guest molecules, which have a higher affinity for the cyclodextrin cavity than they do for the aqueous phase of the reaction mixture. Jambhekar and Breen (Jambhekar SS and Breen P. Drug Disc Today, 2016, 21(2):356-362.) describe ejection of water molecules by the cyclodextrin cavity in favor of the guest molecule.

The review article of Szejtli (Szejtli J. Past, present and future of cyclodextrin research. Pure Appl Chem., 2004, 76(10):1825-1845.) discusses the need for water in the formation of an inclusion complex.

Yoshii et al. (Yoshii H, Kometani T, Furuta T, Watanabe Y, Linko Y Y and Linko P. Formation of inclusion complexes of cyclodextrin with ethanol under anhydrous conditions. Biosci Biotechnol Biochem. 6(11):2166-2170;1998.) describe a method for preparing cyclodextrin complexes with d-limonene in the absence of water. The method is very impractical, being labor-intensive and requiring up to about a forty-fold excess of guest molecule to form an inclusion complex. This large excess of guest molecule requires additional rather aggressive efforts to eliminate adsorbed (as opposed to included) guest molecules. This, in turn, leads to a large loss in the molecule of interest. Furthermore, it is not clear whether or not a cannabinoid would survive the aggressive procedures required to eliminate adsorbed molecules. As the guest molecule is not only the molecule of interest but also likely the more expensive of the two inclusion components, Yoshii's approach is highly inefficient.

### ADVANTAGES OF THE INVENTION

Contrary to prior understanding, it is suggested herein that it is not necessary to sequester an entire hydrophobic cannabinoid compound in the pore of a cyclodextrin molecule (e.g., alpha-cyclodextrin, beta-cyclodextrin, or gamma-cyclodextrin) in order to render it water soluble. Rather, it is possible to render a large molecule like a cannabinoid soluble by sequestering only a portion of the molecule. Without being bound by theory, it is likely that the five-carbon alkane chain common to all cannabinoid molecules is sequestered by the cyclodextrin. Furthermore, it is suggested herein, based upon data using alpha-cyclodextrin, that the binding is a relatively weak binding process, facilitating rapid release of the partially sequestered cannabinoid molecule when it comes in contact with mucosal membranes, and thereby facilitating rapid onset of desired physio- and psychological effects of a cannabinoid.

The pore size of the larger beta-cyclodextrin is appropriate to sequester most if not all of a cannabinoid molecule. However, beta-cyclodextrin is not well tolerated by the human gastrointestinal tract and consequently has a much lower Generally Recognised As Safe (GRAS) threshold than does alpha-cyclodextrin.

Another advantage of alpha-cyclodextrin over the larger beta-cyclodextrin or gamma-cyclodextrin is that alpha-cyclodextrin is the correct size to complex many of the foul-tasting terpenes that tend to be found in cannabinoid extracts. By complexing these terpene molecules, they are rendered both water soluble and tasteless which is of benefit in the preparation of beverages, confectionaries and other edibles.

As provided herein, it has now unexpectedly been found that a cyclodextrin is not required to be dissolved in water or other aqueous solvent in order to form a complex between the hydrophobic cannabinoid and cyclodextrin. Unexpectedly, and indeed surprisingly, for example in light of Jambhekar and Breen (Jambhekar SS and Breen P. Drug Disc Today, 2016, 21(2):356-362.), the phase transfer occurs even between a cannabinoid dissolved in a low molecular weight alcohol (e.g., ethyl alcohol or propyl, including isopropyl alcohol) and a dry powdered alpha-cyclodextrin.

This unexpected and surprising non-aqueous approach, as provided herein, facilitates a simpler and more efficient route to complex formation. This method also is advantageous in avoiding the need to remove water once mixing and complex formation is complete.

### BRIEF DESCRIPTION OF THE DRAWINGS

FIG. 1 depicts the results of adding an overly concentrated Cannabis plant extract to a slurry of α-cyclodextrin in water.

FIG. 2 depicts a commercially-available cannabinoid-containing oil diluted with vegetable oil when first mixed with a slurry of α-cyclodextrin and water.

FIG. 3 depicts a commercially-available cannabinoid-containing oil diluted with vegetable oil mixed with a slurry of α-cyclodextrin in water after gentle mixing for several hours.

FIGS. 4A and 4B depict a comparison between water with (FIG. 4A) or without (FIG. 4B) the addition of a dried Cannabis plant extract composition comprising THC and an aqueous α-cyclodextrin prepared as described herein. FIG. 4A depicts a graduated cylinder containing 100 mL water mixed with 863 mg of a dried Cannabis plant extract composition comprising 1 mg THC and α-cyclodextrin (final concentration, 10 mg/L THC). The concentration of THC in the aqueous mixture was 10 mg/L. FIG. 4B depicts a graduated cylinder containing 100 mL water without the addition of the THC/α-cyclodextrin-containing Cannabis plant extract.

FIG. 5 depicts a pure, white free-flowing powder of alpha-cyclodextrin starting material.

FIG. 6 depicts a viscous, dark green/black, undiluted full spectrum distillate (58% CBD, 2.8% THC, 2.4% CannaBiChromene, 0.8% CannaBiGerol, 0.3% CannaBiDiVarin and 0.2% Other Cannabinoids).

FIG. 7 depicts a CBD full spectrum organic distillate following dilution with ethyl alcohol.

FIG. 8 depicts a damp powder containing a cannabinoid/terpene/α-cyclodextrin mixture following blending.

FIG. 9 depicts a dried cannabinoid-containing powder after evaporation of ethyl alcohol.

FIG. 10 depicts a full spectrum cannabinoid-containing powder dissolved in water at 15 mg/L CBD.

FIG. 11 depicts absorption of cannabinoid complexed with an α-cyclodextrin.

### DESCRIPTION OF THE INVENTION

Described herein is a method for preparing a composition comprising water soluble complexes of α-cyclodextrin and a cannabinoid by mixing one or more cannabinoids with an aqueous α-cyclodextrin-containing composition under conditions wherein the α-cyclodextrin forms complexes with the cannabinoids. Also described herein is an unexpected and surprising method for preparing complexes of a non-aqueous α-cyclodextrin and a cannabinoid by mixing one or more cannabinoids with a non-aqueous α-cyclodextrin-containing composition under conditions wherein the non-aqueous α-cyclodextrin complexes with one or more cannabinoid.

The cannabinoids may be a phytocannabinoids, i.e., naturally-occurring cannabinoids, or synthetically produced cannabinoids referred to hereinafter as “synthetic cannabinoids.” An embodiment of this invention is a method for preparing a Cannabis plant extract-derived composition, by treating a Cannabis plant extract with an aqueous α-cyclodextrin-containing composition under conditions wherein the α-cyclodextrin forms complexes with the cannabinoids and terpenes in the plant extract thereby producing the Cannabis plant extract-derived composition. Another embodiment of this invention is a method for preparing a Cannabis plant extract-derived composition, by treating a Cannabis plant extract with a non-aqueous α-cyclodextrin-containing composition under conditions wherein the non-aqueous α-cyclodextrin forms complexes with the cannabinoids and terpenes in the plant extract thereby producing the Cannabis plant extract-derived composition.

A further embodiment of this invention is a method for preparing a synthetic cannabinoid-derived composition comprising water soluble complexes of α-cyclodextrin and synthetic cannabinoids, by mixing a composition comprising synthetic cannabinoids with an aqueous α-cyclodextrin-containing composition under conditions wherein the α-cyclodextrin forms complexes with the synthetic cannabinoids thereby producing the synthetic cannabinoid-derived composition. Another embodiment of this invention is a method for preparing a synthetic cannabinoid-derived composition comprising complexes of α-cyclodextrin and synthetic cannabinoids, by mixing a composition comprising synthetic cannabinoids with a non-aqueous α-cyclodextrin-containing composition under conditions wherein the non-aqueous α-cyclodextrin forms complexes with the synthetic cannabinoids thereby producing the synthetic cannabinoid-derived composition.

Also described herein is a Cannabis plant extract-derived composition comprising complexes of α-cyclodextrin and at least one cannabinoid, or complexes of α-cyclodextrin and at least one terpene, or both complexes of α-cyclodextrin and at least one cannabinoid and complexes of α-cyclodextrin and at least one terpene. The cannabinoids may be, e.g., THC, CBD, cannabinol, or mixtures thereof. The α-cyclodextrin may be in aqueous or non-aqueous form.

Also described herein is a composition comprising complexes of α-cyclodextrin and at least one synthetic cannabinoid. Synthetic cannabinoids are well known in the art and include, e.g., 1-pentyl-3-(1-naphthoyl)indole (JWH-018), 1-butyl-3-(1-naphthoyl)indole (JWH-073), 1-[2-(4-morpholinyl)ethyl]-3-(1-naphthoyl)indole (JWH-200), 5-(1,1-dimethylheptyl)-2-[(1R,3S)-3-hydroxycyclohexyl]-phenol (CP-47,497), and 5-(1,1-dimethyloctyl)-2-[(1 R, 3 S)-3-hydroxycyclohexyl]-phenol (cannabicyclohexanol; CP-47,497 C8 homologue) and other CBD analogs (see e.g., Morales et al. Frontiers in Pharmacology 28 Jun. 2017 Vol 8, Article 422 p. 1-18). Synthetic cannabinoids also include the FDA-approved compounds dronabinol, a synthetic delta-9-tetrahydrocannabinol (THC), and nabilone, which has a chemical structure similar to THC and is synthetically derived.

The compositions of this invention, e.g., the Cannabis plant extract-derived composition and synthetic cannabinoid-derived compositions comprising complexes of α-cyclodextrin and cannabinoids and/or complexes of α-cyclodextrin and terpenes, exhibit reduced or no odor and/or taste associated with cannabinoids or terpenes, as compared to the starting material, e.g. a Cannabis plant extract or a synthetic cannabinoid. Moreover, the compositions of this invention achieve the effects associated with administration of cannabinoids, e.g., reduction in symptoms of pain or anxiety or both, and/or an increase in feelings of well-being, more rapidly than achieved with the starting material, e.g., the untreated Cannabis plant extract or synthetic cannabinoid. These effects are also prolonged as compared to the effects achieved with the untreated material.

Without wishing to be bound by theory it is contemplated that the α-cyclodextrin and cannabinoid complexes are amphiphilic complexes, having a hydrophilic or polar end and a hydrophobic or nonpolar end, that are absorbed quickly through mucosal membranes such that the cannabinoids are absorbed into the blood stream of the subject more quickly as compared to uncomplexed cannabinoids. The compositions of this invention comprising complexes of α-cyclodextrin and cannabinoids, and/or complexes of α-cyclodextrin and terpenes may be in the form of, e.g., a powder, crystals, granules, an aqueous solution, an aqueous slurry, or an aqueous suspension. The powder, crystals, granules, aqueous solution, uniform aqueous slurry and uniform aqueous suspension offer the added benefit of controlled and consistent dosing of the cannabinoids contained therein.

It has been discovered that α-cyclodextrin renders phytocannabinoids and terpenes in Cannabis plant extracts, and synthetic cannabinoids having aliphatic chains long enough to complex with α-cyclodextrin, soluble in aqueous medium. Without wishing to be bound by theory, it is contemplated that the terpenes form complexes with the α-cyclodextrin and the aliphatic chains of cannabinoids are long enough to form a complex with α-cyclodextrin. Thus, α-cyclodextrin eliminates the terpene taste and odor characteristic of Cannabis plant extracts, e.g., Cannabis oil or Cannabis tar, and renders the cannabinoids and terpenes in the compositions of this invention soluble in an aqueous medium.

The Cannabis plant that serves as the source of the plant extract from which the inventive compositions are derived may be any Cannabis plant, including, e.g., a Cannabis sativa, Cannabis indica, or Cannabis ruderalis, hemp, and strains and hybrid varieties, including crossbred varieties, of such Cannabis plants. In an embodiment, the Cannabis plant is not hemp, e.g., a Cannabis plant having a THC content of less than or equal to 0.2% or less than or equal to 0.3% of the dried flowering portion of the plant. In some embodiments, the extract is from a Cannabis plants having a THC content of the dried flowering portion of the plant of less than or equal to 0.3%. In various embodiments, the Cannabis plant is marijuana. In some embodiments, the extract is from a Cannabis plants having a THC content of the dried flowering portion of the plant of greater than 0.3%. In some embodiments of this invention, the extract is prepared from a Cannabis plant having greater than 0.3% to about 30% THC content of dried flowering portion, and in other embodiments, the THC content of the dried flowering portion of the plant is about 5% to about 30%, or about 5% to about 20%, of the dried flowering portion of the plant.

In addition to the commercially-available Cannabis plant extracts comprising cannabinoids that are suitable for use in the methods of this invention, those of skill in the art recognize that there are a variety of extraction methods, e.g., solvent extraction, and CO₂ extraction, known in the art for producing a Cannabis plant extract comprising cannabinoids, or an extract comprising cannabinoids and terpenes, that are suitable for use in the methods of this invention. Some of the known methods produce Cannabis plant extracts containing high levels of THC, in some instances as high as 80%, 90%, or more (see e.g., Romano and Hazekamp Cannabinoids 2013:1(1):1-11, U.S. Pat. No. 8,895,078, and WO 00/25127, incorporated herein by reference in their entireties). In some embodiments, the Cannabis plant extract is treated to increase the ratio of cannabinoids to terpenes, e.g. by reducing the amount of terpenes and/or increasing the concentration of cannabinoids. For example, the cannabis plant extract may be subjected to purification techniques known in the art, such as distillation, chromatography, or crystallization to obtain a composition high in cannabinoid content with a reduced level of terpenes. A benefit of using an unpurified Cannabis plant extract to produce the composition of this invention is that the resulting composition contains a broad spectrum, if not all, of the medicinally-beneficial cannabinoids of the starting Cannabis plant extract.

One embodiment of the invention is a method for making a Cannabis plant-extract derived composition comprising α-cyclodextrin and a compound selected from cannabinoids, terpenes, or mixtures thereof, the method comprising the steps of: (a) combining a Cannabis plant extract comprising at least one cannabinoid and/or at least one terpene with an aqueous α-cyclodextrin-containing composition, e.g., an aqueous α-cyclodextrin solution, an aqueous α-cyclodextrin slurry, or an aqueous α-cyclodextrin suspension; and (b) mixing the Cannabis plant extract and the aqueous α-cyclodextrin composition for a sufficient time to form a complex between the aqueous α-cyclodextrin and the cannabinoid(s) and/or terpene(s), thereby producing an aqueous mixture comprising complexes of α-cyclodextrin and a compound selected from cannabinoids, terpenes, and/or mixtures thereof. The method may further comprise the step of (c) drying the mixture from step (b), thereby producing a Cannabis plant extract-derived composition comprising complexes of α-cyclodextrin and at least one cannabinoid, or complexes of α-cyclodextrin and at least one terpene, or mixtures of complexes of α-cyclodextrin and at least one cannabinoid and complexes of α-cyclodextrin and at least one terpene, in the form of an odorless and/or tasteless water-soluble powder.

Suitable methods for drying the mixture include evaporating the mixture in dishes or pans, evaporating under reduced pressure and/or ambient temperatures and/or moderately elevated temperatures (e.g., temperatures ranging from 40-150° C.), evaporating in a rotary evaporator, freeze drying or lyophilization, spray drying, or by any other drying method known to a person of skill in the art. This method may also be applied to a synthetic cannabinoid containing composition rather than a Cannabis plant extract to produce a synthetic cannabinoid-derived composition comprising complexes of α-cyclodextrin and one or more synthetic cannabinoids.

Another embodiment of the invention is a method for making a Cannabis plant-extract derived composition comprising α-cyclodextrin and a compound selected from cannabinoids, terpenes, or mixtures thereof, the method comprising the steps of: (a) combining a Cannabis plant extract comprising at least one cannabinoid and/or at least one terpene with a non-aqueous α-cyclodextrin-containing composition, e.g., a non-aqueous α-cyclodextrin powder, crystals, or granules; and (b) mixing the Cannabis plant extract and the non-aqueous α-cyclodextrin composition for a sufficient time to form a complex between the non-aqueous α-cyclodextrin and the cannabinoid(s) and/or terpene(s), thereby producing a non-aqueous mixture comprising complexes of α-cyclodextrin and a compound selected from cannabinoids, terpenes, and/or mixtures thereof. The method may further comprise the step of (c) removing diluents from the mixture of step (b), thereby producing a Cannabis plant extract-derived composition comprising complexes of α-cyclodextrin and at least one cannabinoid, or complexes of α-cyclodextrin and at least one terpene, or mixtures of complexes of α-cyclodextrin and at least one cannabinoid and complexes of α-cyclodextrin and at least one terpene, in the form of an odorless and/or tasteless water-soluble powder. Suitable methods for removing diluents from the mixture include evaporating diluents from the mixture in dishes or pans, evaporating under reduced pressure and/or ambient temperatures and/or moderately elevated temperatures (e.g., temperatures ranging from 40-150° C.), evaporating in a rotary evaporator, freeze drying or lyophilization, spray drying, or by any other drying method known to a person of skill in the art. This method may also be applied to a synthetic cannabinoid containing composition rather than a Cannabis plant extract to produce a synthetic cannabinoid-derived composition comprising complexes of α-cyclodextrin and one or more synthetic cannabinoids.

In an embodiment, the method of (a) combining a Cannabis plant extract comprising at least one cannabinoid and/or at least one terpene with a non-aqueous α-cyclodextrin-containing composition, e.g., a non-aqueous α-cyclodextrin powder, crystals, or granules; and (b) mixing the Cannabis plant extract and the non-aqueous α-cyclodextrin composition for a sufficient time to form a complex between the non-aqueous α-cyclodextrin and the cannabinoid(s) and/or terpene(s) may not require a step of (c) drying the mixture from step (b). Step (c) of removing diluents from the mixture of step (b) may optionally be performed in the non-aqueous embodiment. Advantageously, by using non-aqueous cyclodextrin, step (c) of removing one or more diluents may be advantageously shorter, as compared with the drying of step (c) when aqueous cyclodextrin is used.

The non-aqueous methods may also be applied to a synthetic cannabinoid containing composition rather than a Cannabis plant extract to produce a synthetic cannabinoid-derived composition comprising complexes of α-cyclodextrin and one or more synthetic cannabinoids.

The Cannabis plant extract or synthetic cannabinoids and the aqueous α-cyclodextrin-containing composition are mixed together for a length of time sufficient to form complexes between the α-cyclodextrin and the cannabinoid(s) and/or terpene(s), e.g., at least 5 minutes, at least 15 minutes, at least 30 minutes, at least 45 minutes, at least 1 hour, at least 3 hours, at least 6 hours, or at least 12 hours. The Cannabis plant extract or synthetic cannabinoids and the aqueous α-cyclodextrin containing composition are mixed together under conditions wherein an emulsion is not formed. In one embodiment, the synthetic cannabinoid-derived composition of this invention comprising complexes of α-cyclodextrin and the synthetic cannabinoids is not an emulsion.

The Cannabis plant extract or synthetic cannabinoids and the non-aqueous α-cyclodextrin-containing composition are mixed together vigorously for a length of time sufficient to form complexes between the non-aqueous α-cyclodextrin and the cannabinoid(s) and/or terpene(s), e.g., until the material appears uniform and homogeneous. The Cannabis plant extract or synthetic cannabinoids and the non-aqueous α-cyclodextrin containing composition are mixed together under conditions wherein a complex is formed. By way of non-limiting example, the cannabinoid is diluted in alcohol (e.g., ethyl or isopropy), followed by adding the diluted cannabinoid to dry α-CD, followed by vigorous blending in a food processor or blender. In an embodiment, the blended materials are incubated in a sealed container at about ambient temperature, about 50° C., about 60° C., or about 70° C. for about 5 minutes, about 15 minutes, about 30 minutes, about 45 minutes, about 1 hour, about 90 minutes, about 2 hours, about 2.5 hours, about 3 hours, about 3.5 hours, about 4 hours before the seal is removed. In another preferred embodiment, the blended materials do not require incubation after blending.

Following blending and any incubation, the alcohol diluent is allowed to evaporate at any time and temperature sufficient to achieve evaporation of the diluent without alteration of the chemical composition of the blended materials. In an embodiment, evaporation is conducted at about 50° C., about 60° C., or about 70° C. for about 2-4 hours, about 1-2 hours, or about 30 minutes -1 hour. In a preferred embodiment, evaporation is conducted at about 60° C. for about 1-2 hours.

Suitable aqueous α-cyclodextrin-containing compositions for mixing with the Cannabis plant extract or synthetic cannabinoids in the methods described herein comprise at least 15% w/v α-cyclodextrin. Preferable α-cyclodextrin slurries or suspensions comprise at least 20% w/v α-cyclodextrin. Preferable α-cyclodextrin slurries or suspensions comprise at least 25% w/v α-cyclodextrin. Suitable aqueous α-cyclodextrin slurries or suspensions comprise an amount of α-cyclodextrin that exceeds the solubility of α-cyclodextrin in the solution such that non-solubilized α-cyclodextrin is present in the slurry or suspension. In an embodiment of this invention, the aqueous α-cyclodextrin compositions for mixing with the Cannabis plant extract or synthetic cannabinoids comprise about 15% w/v to about 40% w/v α-cyclodextrin, about 20% w/v to about 35% w/v α-cyclodextrin or about 20% to about 30% w/v α-cyclodextrin.

Suitable non-aqueous α-cyclodextrin-containing compositions for mixing with the Cannabis plant extract or synthetic cannabinoids in the methods described herein comprise a concentration of at least about 2000 grams of α-cyclodextrin per liter of Cannabis extract/synthetic cannabinoid mixture. Other non-aqueous α-cyclodextrin powders, crystals, or granules comprise a concentration of at least about 2250 grams of α-cyclodextrin per liter of Cannabis extract/synthetic cannabinoid mixture. Preferable α-cyclodextrin powders, crystals, or granules may also comprise at least about 2500 grams of non-aqueous α-cyclodextrin per liter of Cannabis extract/synthetic cannabinoid mixture. In other embodiments of this invention, the non-aqueous α-cyclodextrin compositions for mixing with the Cannabis plant extract or synthetic cannabinoids comprise at least about 2750 grams, at least about 3000 grams, at least about 3500 grams, at least about 4000 grams, at least about 5000 grams, at least about 7500 grams, or at least about 10,000 grams of α-cyclodextrin per liter of other components. In a further embodiment, the non-aqueous α-cyclodextrin compositions for mixing with the Cannabis plant extract or synthetic cannabinoids comprise more than 10,000 grams of α-cyclodextrin to a liter of other components.

It is recognized that the amount of α-cyclodextrin to be mixed with the Cannabis plant extract or synthetic cannabinoids to complex with the cannabinoids and terpenes may be varied depending on the characteristics of the starting material, e.g., its viscosity, its solid content, characteristics of its other solid components, its cannabinoid content, or its terpene content, and the desired characteristics of the composition produced by the methods described herein, e.g., a solution, suspension, slurry, powder, crystals, or granules.

In an embodiment of the invention the ratio of aqueous α-cyclodextrin to the Cannabis plant extract is at least about 70:1 weight/weight (w/w), at least about 80:1 w/w, at least about 85:1 w/w, at least about 95:1 w/w, or at least about 100:1 w/w, at least about 120:1, and any and all ranges therebetween.

In another embodiment of the invention the ratio of non-aqueous α-cyclodextrin to the Cannabis plant extract is at least about 2:1 weight/weight (w/w), at least about 10:1 w/w, at least about 20:1 w/w, at least about 50:1 w/w, at least about 100:1 w/w or at least about 200:1 w/w, at least about 300:1, at least about 500:1, at least about 750:1, at least about 1000:1, at least about 1500:1, at least about 2000:1, at least about 2500:1, at least about 3000:1, at least about 4000:1, at least about 5000:1, at least about 7500:1, at least about 10,000:1, and any and all ranges therebetween. In a further embodiment, the ratio of non-aqueous α-cyclodextrin to the Cannabis plant extract is greater than 10,000:1 weight/weight.

In one embodiment of the invention, the aqueous α-cyclodextrin-containing composition, e.g. a solution, slurry, or suspension, is combined with the Cannabis plant extract or synthetic cannabinoids in an amount and mixed for a time and at a temperature such that at least 80%, at least 85%, at least 90%, at least 95%, at least 99%, or at least 100% of the cannabinoids are complexed with the α-cyclodextrin. In another embodiment, a non-aqueous α-cyclodextrin-containing composition, e.g., a solid cyclodextrin compound, is combined with the Cannabis plant extract or synthetic cannabinoids in an amount and mixed for a time and at a temperature such that at least 80%, at least 85%, at least 90%, at least 95%, at least 99%, or at least 100% of the cannabinoids are complexed with the non-aqueous α-cyclodextrin.

In one embodiment of the invention, the aqueous α-cyclodextrin-containing composition, e.g., a solution, slurry, or suspension, is combined with the Cannabis plant extract in an amount such that at least 80%, at least 85%, at least 90%, at least 95%, at least 99%, or at least 100% of the terpenes are complexed with the α-cyclodextrin. In another embodiment, a non-aqueous α-cyclodextrin-containing solid composition, e.g., powder, crystalline or granule cyclodextrin compound, is combined with the Cannabis plant extract in an amount such that at least 80%, at least 85%, at least 90%, at least 95%, at least 99%, or at least 100% of the terpenes are complexed with the solid α-cyclodextrin.

In one embodiment of the invention, the aqueous α-cyclodextrin-containing composition, e.g., solution, slurry, or suspension, is combined with the Cannabis plant extract or synthetic cannabinoids in an amount such that at least 80%, at least 85%, at least 90%, at least 95%, at least 99%, or at least 100% of the cannabinoids and terpenes are complexed with the α-cyclodextrin. In another embodiment, a non-aqueous α-cyclodextrin-containing composition, e.g., a solid cyclodextrin compound (e.g., powder, crystal, or granule), is combined with the Cannabis plant extract or synthetic cannabinoids in an amount such that at least 80%, at least 85%, at least 90%, at least 95%, at least 99%, or at least 100% of the cannabinoids and terpenes are complexed with the solid α-cyclodextrin.

In an embodiment of this invention, to achieve the Cannabis plant extract-derived composition or synthetic cannabinoid-derived composition of this invention having essentially no odor or taste typically associated with a cannabinoid, the ratio of aqueous α-cyclodextrin to cannabinoids in the extract or synthetic cannabinoid is greater than about 70:1 w/w, greater than 85:1 w/w, greater than 90:1 w/w, greater than 95:1 w/w, greater than about 100:1 w/w and greater than about 120:1 w/w and ranges there between.

In another embodiment of the invention, to achieve the Cannabis plant extract-derived composition or synthetic cannabinoid-derived composition of this invention having essentially no odor or taste typically associated with a cannabinoid, the ratio of non-aqueous α-cyclodextrin to cannabinoids in the extract or synthetic cannabinoid is greater than about 10:1 weight/weight (w/w), greater than about 20:1 w/w, greater than about 50:1 w/w, greater than about 100:1 w/w, or greater than about 200:1 w/w, greater than about 300:1, greater than about 500:1, greater than about 750:1, greater than about 1000:1, greater than about 1500:1, greater than about 2000:1, greater than about 2500:1, greater than about 3000:1, greater than about 4000:1, greater than about 5000:1, greater than about 7500:1, greater than about 10,000:1, and any and all ranges therebetween. In a further embodiment, the ratio of non-aqueous α-cyclodextrin to the Cannabis plant extract is greater than 10,000:1 weight/weight.

U.S. Patent No. 2017/0224842 (Czap) describes mixing cyclodextrin with cannabinoids from partially purified, low terpene containing, hemp oil. Unlike the Czap formulations, which are formulated with amylase-like enzymes that hydrolyze the cyclodextrins, the Cannabis plant extract-derived compositions and synthetic cannabinoid-derived composition of this invention do not require an amylase-like enzyme to be effective or to achieve their desired results. Thus, an aspect of this invention is a Cannabis plant extract-derived compositions or synthetic cannabinoid-derived composition comprising complexes of α-cyclodextrin and a compound selected from cannabinoids, terpenes, or mixtures thereof, wherein the compositions do not comprise an amylase or an amylase-like enzyme, e.g., a cyclodextrinase, maltogenic amylase, or neopullulanase, that is formulated so that the cyclodextrin-degrading activity of the enzyme is activated on delivery of the composition to a subject.

In one embodiment, the plant extract-derived compositions of this invention comprise α-cyclodextrin complexes comprising THC, CBD, cannabinol, or mixtures thereof. In an embodiment of the invention, the cannabinoids in complex with α-cyclodextrin are selected from the group consisting of THC, CBD, cannabinol, and mixtures thereof.

Preferably, the Cannabis plant extract and synthetic cannabinoids (or compositions comprising synthetic cannabinoids) have a consistency that facilitates mixing with the aqueous or non-aqueous α-cyclodextrin-containing composition. For example, the Cannabis plant extract and synthetic cannabinoids may have the consistency of a free-flowing oil. Thus, in an embodiment of this invention, the Cannabis plant extract or synthetic cannabinoid is mixed with a solvent to facilitate its mixing with an aqueous α-cyclodextrin-containing composition. The solvent may be for example water, an oil, or an alcohol.

In the case of mixing with a non-aqueous, α-cyclodextrin-containing composition, the Cannabis plant extract (or compositions comprising synthetic cannabinoids) may be in a solid form, without solvent. Alternatively, the Cannabis plant extract (or compositions comprising synthetic cannabinoids) may be contained in any non-aqueous solvent, including for example, ethyl alcohol, propyl alcohol, isopropyl alcohol, or butyl alcohol.

In an embodiment, the amount of solvent added to the Cannabis plant extract or synthetic cannabinoid is sufficient such that the Cannabis plant extract or synthetic cannabinoid is free-flowing. The amount of solvent added to the Cannabis plant extract or synthetic cannabinoid may be up to 95% v/v of the total volume of the solvent and Cannabis plant extract mixture. The amount of solvent added to the Cannabis plant extract or synthetic cannabinoid is preferably up to 75% v/v. The amount of solvent added to the Cannabis plant extract or synthetic cannabinoid is more preferably up to 50% v/v. The extract or synthetic cannabinoid may be mixed with the solvent before or after, preferably before, combining the extract or synthetic cannabinoid with an aqueous α-cyclodextrin-containing composition. Preferably, the solvent is mixed with the extract or synthetic cannabinoid before combining the extract or synthetic cannabinoid with the aqueous cyclodextrin-containing composition and then mixing the Cannabis plant extract or synthetic cannabinoid and the aqueous cyclodextrin-containing composition for a sufficient time to form complexes between the α-cyclodextrin and the cannabinoid(s) and/or terpene(s). The aqueous α-cyclodextrin-containing composition for use in the method may be an aqueous α-cyclodextrin-containing solution, slurry, or suspension. The non-aqueous α-cyclodextrin-containing composition for use in the method may be in the form of a powder, crystals, granules, or any other solid form.

Suitable oils for mixing with the Cannabis plant extract or synthetic cannabinoid include non-toxic oils, such as edible oils, macerated oils, essential oils, and pharmaceutically acceptable oils. Examples of suitable edible oils include coconut oil, corn oil, olive oil, palm oil, cottonseed oil, peanut oil, rapeseed oil, safflower oil, sunflower oil, sesame oil, soybean oil, almond oil, pumpkin or squash seed oil, Brazil nut oil, cashew oil, hazelnut oil, macadamia nut oil, pecan oil, pine nut oil, pistachio oil, walnut oil, citrus oils, carob oil, cocoa butter, shea butter, hemp oil, flaxseed oil, grapeseed oil, other seed oils. Examples of suitable macerated oils include arnica oil, calendula oil, carrot oil, chickweed oil, comfrey oil, galangal oil, horse chestnut oil, linden blossom oil, marigold oil, meadowsweet oil, mullein oil, sea weed oil, and St. John's Wort oil. Examples of suitable essential oils include bergamot oil, camphor oil, peppermint oil, Cannabis flower essential oil, cardamom seed oil, cinnamon oil, citronella oil, coffee oil, eucalyptus oil, garlic oil, fennel seed oil, fenugreek oil, ginger oil, henna oil, jasmine oil, rose oil, rosehip oil, lavender oil, ylang-ylang oil, wintergreen oil, turmeric oil, thyme oil. Examples of pharmaceutically acceptable oils include ethyl butyrate, ethyl caprylate, ethyl oleate, triglycerides, and soybean oil.

Suitable alcohols for mixing with the Cannabis plant extract or synthetic cannabinoid include alcohols that are suitable for food and/or natural products and/or pharmaceutical production. Examples of suitable alcohols are ethanol and isopropanol. The amount of alcohol added to the Cannabis plant extract or synthetic cannabinoid is sufficient to make the Cannabis plant extract or synthetic cannabinoid free flowing. The amount of alcohol added to the Cannabis plant extract or synthetic cannabinoid may be up to 95% v/v, 90% v/v, 85% v/v, 80% v/v, 75% v/v, 70% v/v, 65% v/v, 60% v/v, 55% v/v, 50% v/v, or 45% v/v of the total volume of the alcohol and Cannabis plant extract mixture. The Cannabis plant extract or synthetic cannabinoid may be mixed with the alcohol before or after combining the extract or synthetic cannabinoid with an aqueous or non-aqueous α-cyclodextrin-containing composition. Preferably, the alcohol is mixed with the extract or synthetic cannabinoid before mixing the extract or synthetic cannabinoid with the aqueous α-cyclodextrin-containing composition, e.g., an aqueous solution, slurry, or suspension or the non-aqueous α-cyclodextrin-containing composition, e.g., powder, crystals, or granules. The Cannabis plant extract or synthetic cannabinoid may be mixed with an alcohol in a 1:15 w/v, 1:10 w/v, 1:5 w/v, 1:2 w/v ratio of extract to alcohol, and ranges there between.

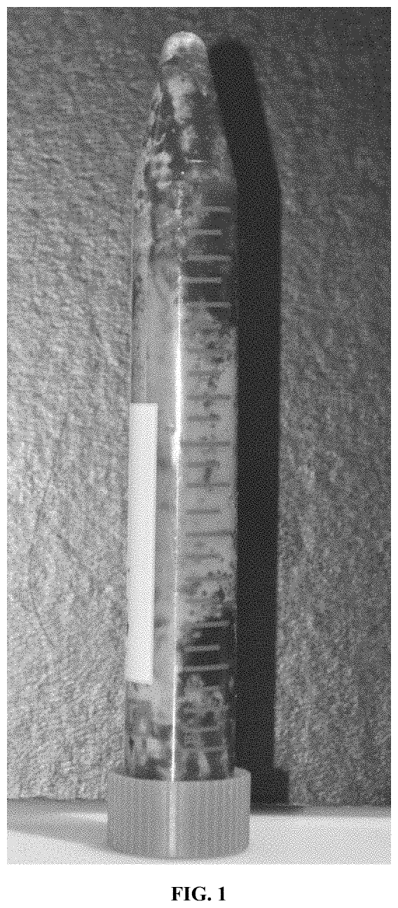

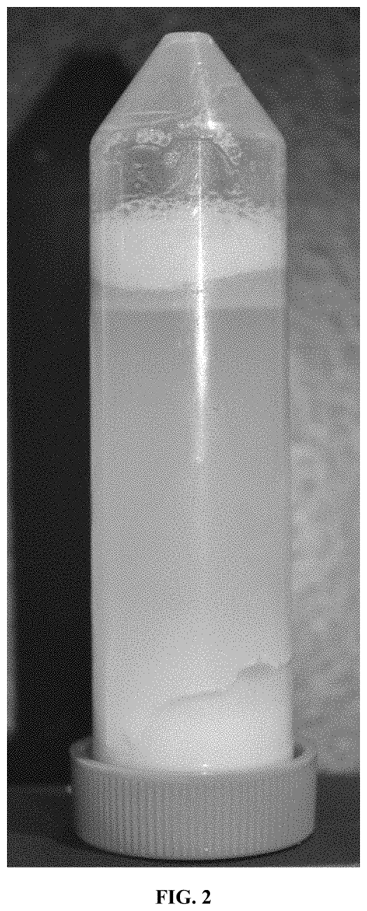

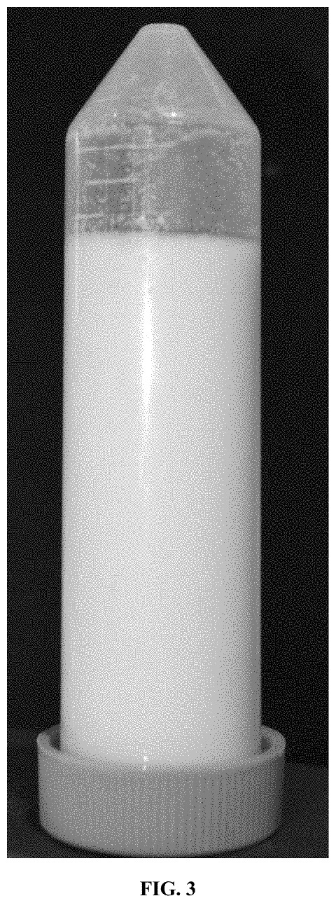

The effects of the consistency and/or viscosity of the plant extract in the methods of this invention are shown in FIGS. 1-3. As shown in FIG. 1, adding an overly concentrated tar-like cannabinoid-containing extract to an α-cyclodextrin slurry results in the tar-like material not being suspended in an aqueous α-cyclodextrin slurry. Instead, the tar-like material congeals into clumps of material that stick to the walls of the container (e.g., a tube). The clumps of material remain unchanged even after extensive mixing. In contrast, FIG. 2 shows a tube containing a commercially-available cannabinoid-containing oil diluted with vegetable oil when it is first mixed with an aqueous α-cyclodextrin containing slurry. As can be seen, undissolved α-cyclodextrin rests at the bottom of the tube while the middle portion of the tube contains an aqueous layer comprising both dissolved α-cyclodextrin and suspended undissolved α-cyclodextrin. The cannabinoid-containing oil appears to be distributed between the undissolved α-cyclodextrin layer and the top of the aqueous layer. Gentle mixing is used in order to avoid the formation of froth, as seen at the top of the container, and to avoid the formation of an emulsion. After gently mixing the tube for several hours, the α-cyclodextrin/oil slurry mixture appears homogeneous and has a uniform off-white color (FIG. 3). Due to the gentle mixing, an emulsion is not formed and there is no froth at the top of the container. The aqueous mixture in the container is ready for using as a slurry in an edible product, e.g., a cupcake, and/or for drying, e.g., into a powder.

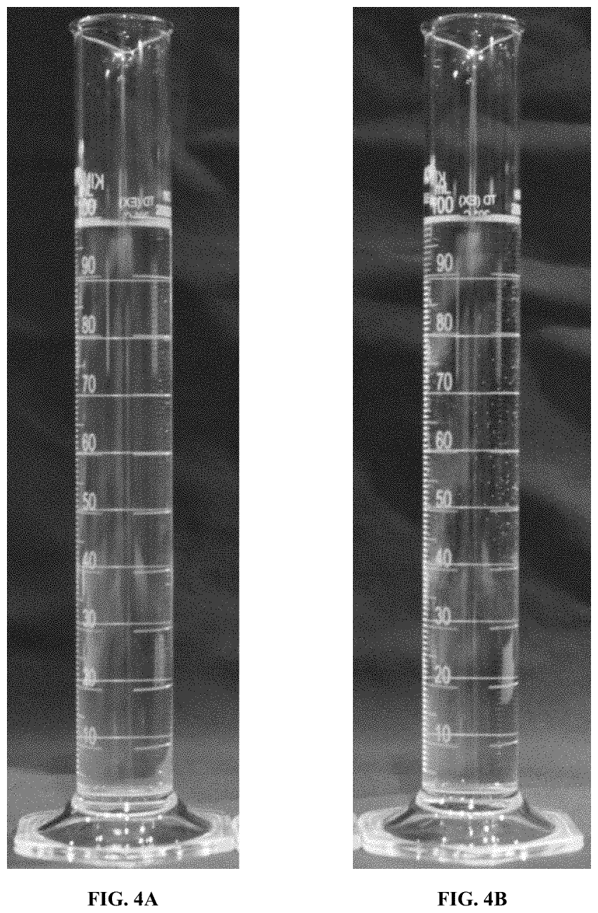

An embodiment of this invention is a Cannabis plant extract-derived composition or synthetic cannabinoid-derived composition comprising complexes of cannabinoids and aqueous or non-aqueous α-cyclodextrin, where the complexes are water soluble. FIG. 4A depicts a graduated cylinder containing a mixture of 100 mL water and 863 mg of a dried Cannabis plant extract-derived composition of this invention comprising 1 mg THC in complexes with α-cyclodextrin and FIG. 4B depicts a graduated cylinder with just 100 ml water. The mixture in FIG. 4A and the water in FIG. 4B are indistinguishable. The Cannabis plant extract composition combined with water depicted in FIG. 4A was prepared from Cannabis plant distillate.

An aspect of this invention is a Cannabis plant extract-derived composition comprising water soluble complexes of α-cyclodextrin and terpenes; α-cyclodextrin and THC; α-cyclodextrin and CBD, or α-cyclodextrin and cannabinol, or mixtures of such complexes. Another aspect of the invention is a Cannabis plant extract-derived composition comprising complexes selected from the group consisting of α-cyclodextrin and terpenes, α-cyclodextrin and THC, α-cyclodextrin and CBD, α-cyclodextrin and cannabinol, and mixtures of such complexes that are water soluble. An aspect of this invention is a synthetic cannabinoid-derived composition comprising water soluble complexes of α-cyclodextrin and one or more synthetic cannabinoids. Complexes of α-cyclodextrin and terpenes; α-cyclodextrin and THC; α-cyclodextrin and CBD, or α-cyclodextrin and cannabinol, or mixtures of such complexes may themselves be water soluble or may be soluble in alcohol, oil, or any other liquids, or combination of liquids, regardless of whether aqueous α-cyclodextrin or non-aqueous α-cyclodextrin is used to form the complex.

In one embodiment of the invention, the Cannabis plant-derived composition or synthetic cannabinoid-derived composition of this invention is an aqueous solution, an aqueous slurry, or an aqueous suspension comprising complexes of α-cyclodextrin and cannabinoid(s) and/or complexes of α-cyclodextrin and terpene(s) or mixtures of such complexes. In another embodiment of the invention, the Cannabis plant-derived composition or synthetic cannabinoid-derived composition of this invention is a solution, slurry, or suspension comprising complexes of α-cyclodextrin and cannabinoid(s) and/or complexes of α-cyclodextrin and terpene(s) or mixtures of such complexes in a non-aqueous solvent.

In a further embodiment, the Cannabis plant-derived composition or synthetic cannabinoid-derived composition of this invention is a non-aqueous solid comprising complexes of α-cyclodextrin and cannabinoid(s) and/or complexes of α-cyclodextrin and terpene(s) or mixtures of such complexes.

In one embodiment of the invention, the Cannabis plant-derived composition or synthetic cannabinoid-derived composition of this invention is a water-soluble powder, crystals, or granules comprising complexes of α-cyclodextrin and cannabinoid(s) and/or complexes of α-cyclodextrin and terpene(s) or mixtures of such complexes.

An embodiment of the invention is a liquid comprising the Cannabis plant extract-derived composition or synthetic cannabinoid-derived composition described herein comprising complexes of α-cyclodextrin and cannabinoid(s) and/or complexes of α-cyclodextrin and terpene(s) or mixtures of such complexes. The liquid may be, for example, a water (including flavored water), a tea, a coffee, a juice, a smoothie, any carbonated beverage (e.g., a carbonated water or soft drink), a sports or other rehydration drink, a mocktail, or an alcoholic beverage (e.g., beer, wine, wine cooler, cocktail, or liquor).

Another aspect of the invention is a food product comprising a Cannabis plant extract-derived composition or synthetic cannabinoid-derived composition of this invention such that the food comprises complexes of α-cyclodextrin and at least one cannabinoid and/or complexes of α-cyclodextrin and at least one terpene, or mixtures of such complexes. In one embodiment, the food product of the invention may comprise a sweetening agent, a coloring agent and/or a flavoring agent known in the art.

In one embodiment, the food product is a farinaceous food product or a confection. Examples of a farinaceous food products include, e.g., cake, muffin, brownie, cookie, cracker, doughnut, or biscuit. Examples of a confection include e.g., caramel, chocolate, nougat, chewing gum, toffee, lozenge, fondant, halvah, jelly, gelatin, candies, e.g., gummies (e.g., gummy bears), suckers or lollipops, licorice, marshmallow, taffy, or marzipan.

In another embodiment, the food product is a non-farinaceous food product. Examples of a non-farinaceous food product include e.g., a dairy product, a meat product, a fruit product, or a vegetable product. Preferred examples of a non-farinaceous food product include beverages, such as tea, herbal tea, coffee, juice, bottled water, carbonated beverages, smoothies, sports or other rehydration drinks, mocktails or alcoholic beverages.

While Cannabis plant-derived compositions and synthetic cannabinoid-derived compositions of this invention can be added to the food products in any amount, it is understood that a preferred amount would be such that the organoleptic properties between the food product with and without the Cannabis plant-derived compositions or synthetic cannabinoid-derived compositions are not significantly different.

The Cannabis plant extract-derived composition or synthetic cannabinoid-derived composition of this invention may be mixed with an edible oil, e.g., include coconut oil, corn oil, olive oil, palm oil, cottonseed oil, peanut oil, rapeseed oil, safflower oil, sunflower oil, sesame oil, soybean oil, almond oil, pumpkin or squash seed oil, Brazil nut oil, cashew oil, hazelnut oil, macadamia nut oil, pecan oil, pine nut oil, pistachio oil, walnut oil, citrus oils, carob oil, cocoa butter, shea butter, hemp oil, flaxseed oil, grapeseed oil, other seed oils.

Another aspect of this invention is a method for increasing the content of THC and/or CBD and/or synthetic cannabinoid in a food product by adding the Cannabis plant-derived composition or synthetic cannabinoid-derived composition of this invention to a food product in an amount sufficient to increase the THC and/or CBD and/or synthetic cannabinoid content of the food product.

The Cannabis plant-derived composition or synthetic cannabinoid-derived composition of this invention may be added to the food product at any stage of its preparation. For example, a Cannabis plant extract-derived composition or synthetic cannabinoid-derived composition described herein may be mixed with the food product's ingredients so that it is distributed throughout the food product and the product may then be cooked, baked, grilled, or fried, or the composition may be mixed or added to the final food product after cooking, baking, grilling or frying. In some instances, a Cannabis plant extract-derived composition and synthetic cannabinoid-derived composition of this invention may be applied to the surface of the food product, e.g., as a glaze, icing, sprinkling, or coating, or the composition may be simply sprinkled on or mixed with the food product, e.g., stirred into a beverage, e.g., a tea, cocoa, coffee, juice, smoothie, or alcoholic beverage.

Another embodiment of the invention is a topical formulation comprising a Cannabis plant extract-derived composition of this invention. An embodiment of this invention is a topical formulation comprising a synthetic, cannabinoid-derived composition of the invention. The topical formulation may be, for example, a lotion, an oil, a topical gel, a spray, an ointment, or a cream comprising the Cannabis plant extract-derived composition or synthetic cannabinoid-derived composition of this invention comprising complexes of α-cyclodextrin and at least one cannabinoid or complexes of α-cyclodextrin and at least one terpene, or comprising mixtures of such complexes.

The topical formulation may include a compound which enhances absorption or penetration of the α-cyclodextrin complexes through the skin. Typical carriers include alcohol, water, mineral oil, liquid petrolatum, white petrolatum, glycerin, dimethylsulfoxide, polyethylene glycol and propylene glycol.

The Cannabis plant extract-derived composition or synthetic cannabinoid-derived composition of this invention may be added to the topical formulation at any stage of its preparation, e.g., the Cannabis plant extract-derived composition or synthetic cannabinoid-derived composition may be mixed with the topical formulation's ingredients and then mixed together or the Cannabis plant extract-derived composition or synthetic cannabinoid-derived composition may be mixed or added to the final topical formulation. The Cannabis plant extract-derived composition or synthetic cannabinoid-derived composition of this invention may be mixed with an oil suitable for topical application, e.g., an essential oil, e.g., bergamot oil, camphor oil, peppermint oil, Cannabis flower essential oil, cardamom seed oil, cinnamon oil, citronella oil, coffee oil, eucalyptus oil, garlic oil, fennel seed oil, fenugreek oil, ginger oil, henna oil, jasmine oil, rose oil, rosehip oil, lavender oil, ylang-ylang oil, wintergreen oil, turmeric oil, or thyme oil.

In one embodiment, the topical formulation of this invention comprises complexes of terpenes. In another embodiment, the topical formulation of this invention comprises complexes of α-cyclodextrin and cannabinoids, e.g., α-cyclodextrin and THC, α-cyclodextrin and CBD, α-cyclodextrin and cannabinol, or α-cyclodextrin and one or more synthetic cannabinoids, or mixtures thereof, where any of the complexes of cannabinoids may also optionally comprise complexes of terpenes. In one embodiment, the topical formulation comprises complexes selected from the group consisting of THC-α-cyclodextrin complexes, CBD-α-cyclodextrin complexes, or cannabinol-α-cyclodextrin complexes, and mixtures thereof. In one embodiment, the topical formulation comprises complexes of-α-cyclodextrin in complex with one or more of 1-pentyl-3-(1-naphthoyl)indole (JWH-018), 1-butyl-3-(1-naphthoyl)indole (JWH-073), 1-[2-(4-morpholinyl)ethyl]-3-(1-naphthoyl)indole (JWH-200), 5-(1,1-dimethylheptyl)-2-[(1R,3S)-3-hydroxycyclohexyl]-phenol (CP-47,497), and 5-(1,1-dimethyloctyl)-2-[(1R,3S)-3-hydroxycyclohexyl]-phenol (cannabicyclohexanol; CP-47,497 C8 homologue) and other CBD analogs (see e.g., Morales et al. Frontiers in Pharmacology 28 Jun. 2017 Vol 8, Article 422 p. 1-18), dronabinol, or nabilone. In one embodiment, the topical formulation comprises complexes of-α-cyclodextrin in complex with one or more synthetic cannabinoids selected from the group consisting of 1-pentyl-3-(1-naphthoyl)indole (JWH-018), 1-butyl-3-(1-naphthoyl)indole (JWH-073), 1-[2-(4-morpholinyl)ethyl]-3-(1-naphthoyl)indole (JWH-200), 5-(1,1-dimethylheptyl)-2-[(1R,3S)-3-hydroxycyclohexyl]-phenol (CP-47,497), and 5-(1,1-dimethyloctyl)-2-[(1R,3S)-3-hydroxycyclohexyl]-phenol (cannabicyclohexanol; CP-47,497 C8 homologue), dronabinol, and nabilone.

An aspect of the invention is a method of alleviating pain in a subject in need thereof, comprising administering to the subject a therapeutically effective amount of a Cannabis plant-derived composition or synthetic cannabinoid-derived composition of the invention described herein, comprising complexes of α-cyclodextrin and at least one cannabinoid (e.g., THC, CBD, and/or cannabinol or a synthetic cannabinoid). The Cannabis plant-derived composition administered may also comprise complexes of α-cyclodextrin and at least one terpene. A therapeutically effective amount of the Cannabis plant-derived composition or the synthetic cannabinoid-derived composition in this embodiment is an amount sufficient to alleviate (e.g., reduce or eliminate) pain in the subject in need thereof. The subject in need thereof is a subject, e.g. a human, non-human primate, horse, cow, sheep, pig, goat, dog, cat, rat, or mouse, suffering from pain.

An aspect of the invention is a method of alleviating anxiety in a subject in need thereof, comprising administering a therapeutically effective amount of a Cannabis plant-derived composition or synthetic cannabinoid-derived composition of the invention described herein comprising complexes of α-cyclodextrin and at least one terpene and/or α-cyclodextrin and at least one cannabinoid (e.g., THC, CBD, and/or cannabinol, or synthetic cannabinoid) to the subject. A therapeutically effective amount of the composition of this invention is an amount sufficient to alleviate (e.g., reduce or eliminate) anxiety in the subject in need thereof. The subject in need thereof is a subject, e.g. a human, non-human primate, horse, cow, sheep, pig, goat, dog, cat, rat, or mouse, suffering from anxiety.

An aspect of the invention is a method of increasing feelings of well-being and relaxation in a subject comprising administering a sufficient amount of a Cannabis plant-derived composition or synthetic cannabinoid-derived composition of the invention comprising complexes of α-cyclodextrin and at least one terpene and/or α-cyclodextrin and at least one cannabinoid (e.g., THC, CBD, and/or cannabinol, or synthetic cannabinoid) to the subject. A sufficient amount of the compositions of this invention is an amount sufficient to increase feelings of well-being and relaxation in the subject.

Another aspect of the invention is a method of treating a subject, e.g. a human, a non-human primate, a horse, cow, sheep, pig, goat, dog, cat, rat, or mouse, having a disorder having symptoms that are alleviated (e.g., reduced or eliminated) by treatment with a cannabinoid. The method comprises administering a therapeutically effective amount of a Cannabis plant-derived composition or synthetic cannabinoid-derived composition of the invention comprising complexes of α-cyclodextrin and at least one terpene and/or α-cyclodextrin and at least one cannabinoid (e.g., THC, CBD, and/or cannabinol and/or a synthetic cannabinoid) to the subject having the disorder. In an aspect of the invention, the composition of this invention administered to the subject comprises complexes of α-cyclodextrin and cannabinoids (e.g., THC, CBD, cannabinol, and synthetic cannabinoids).

Examples of disorders that can be treated with the compositions described herein include trigeminal neuralgia, agitation, Alzheimer's dementia, anxiety disorders, jet lag, depression, anorexia, Attention Deficit Disorder (ADD) with or without hyperactivity, Autism Spectrum Disorder (ASD), cachexia, cancer, chronic cluster headaches, Crohn's disease, convulsions, epilepsy, gastrointestinal tract distress (e.g., heartburn indigestion, stomach ache), glaucoma, HIV/AIDS, Huntington's disease, inflammation (chronic or acute), inflammatory bowel disease, irritable bowel syndrome (IBS) with or without diarrhea or constipation, migraine headaches whether hormone mediated or not, muscle spasms, nausea, neurodegenerative diseases including Lou Gehrig's disease, all forms of pain (e.g., acute, chronic, neuropathic, trigeminal nerve pain, pain associated with or accompanying migraine headaches and cancer), Parkinson's disease and Parkinsonian-type symptoms, spinal-cord injuries, postmenstrual syndrome (PMS), Post-Traumatic Stress Disorder (PTSD), seasonal affective disorder (SAD), seizures, seizures associated with Lennox-Gastaut syndrome or Dravet syndrome, sleep disorders such as insomnia, spasticity from multiple sclerosis, ulcerative colitis, and vomiting. An embodiment of this invention is a method for treating a subject, e.g. a human, having cancer, chronic cluster headaches, Crohn's disease, convulsions, epilepsy, glaucoma, migraine headaches whether hormone mediated or not, nausea, or trigeminal nerve pain by administering to the subject a therapeutically effective amount of a Cannabis plant extract-derived composition or synthetic cannabinoid-derived composition of this invention.

Treatment of a disorder as described herein refers to reducing or eliminating one or more symptoms of the disorder. A therapeutically effective amount of a composition of this invention as described herein for treating a disorder is an amount sufficient to reduce or eliminate one or more symptoms of the disorder. For example, if a subject has a disorder that is associated with pain, a therapeutically effective amount is an amount sufficient to reduce or eliminate the pain. For example, if the disorder is an anxiety disorder a therapeutically effective amount is an amount sufficient to reduce or eliminate anxiety.

A therapeutically effective amount of a Cannabis plant-derived composition or synthetic cannabinoid-derived composition of the invention described herein, comprising complexes of α-cyclodextrin and at least one cannabinoid (e.g., THC, CBD, and/or cannabinol or a synthetic cannabinoid) administered to a subject, e.g. a subject having anxiety, pain or another disorder having symptoms that are alleviated by treatment with a cannabinoid, may be administered to the subject in any suitable form, e.g., a tablet, capsule, pill, elixir, wafer, beverage, or a food product.

The compositions of this invention may be administered to the subject by any suitable method, e.g., orally (swallowed), sublingually (under the tongue), transdermally, transmucosally, intranasally, topically, or rectally, in an amount suitable to achieve the desired effects.

It has been found that the cannabinoids and/or terpenes complexed with α-cyclodextrin in the compositions of this invention are absorbed more rapidly through the mucosal membranes of a subject than are cannabinoids and/or terpenes that are not complexed with α-cyclodextrin. Moreover, the effects of the cannabinoids in the α-cyclodextrin complexes in the Cannabis plant extract-derived compositions and synthetic cannabinoid-derived compositions of this invention are prolonged as compared to the effects of the cannabinoids in the untreated Cannabis plant extracts or synthetic cannabinoids. Without wishing to be bound by theory, it is contemplated that the α-cyclodextrin and cannabinoid complexes are amphiphilic and are thereby absorbed more readily through a mucosal membrane. It is also contemplated that at least a portion of the complexes are delivered to the subject's stomach and intestines where the cannabinoids are released, thereby prolonging their effect on the subject. An embodiment of this invention is a composition comprising cannabinoids formulated to enhance absorption of the cannabinoids through mucosal membranes of a subject, and/or to prolong the effect of the cannabinoids on the subject, wherein the cannabinoids are in the form of complexes with α-cyclodextrin.

An embodiment of this invention is a method for enhancing the absorption of cannabinoids and/or terpenes in a Cannabis plant extract or absorption of synthetic cannabinoids through mucosal membrane of a subject by mixing the Cannabis plant extract or synthetic cannabinoids with an aqueous or a non-aqueous α-cyclodextrin-containing composition under conditions suitable to form complexes of α-cyclodextrin and the cannabinoids and/or terpenes, or both. The resulting composition comprising the complexes of α-cyclodextrin and the cannabinoids and/or terpenes, or both may be administered to the subject or the complexes in the mixture may then be collected, e.g., by filtering the mixture, or drying the mixture, or in the case of a non-aqueous α-cyclodextrin-containing composition simply collecting the mixture by spoon, spatula, or other device or tool. The resulting composition comprising the complexes may be diluted, mixed, or added directly to a food or beverage product and may be administered to the subject.

### EXAMPLES

### Example 1—THC-Containing Cannabis Powders

THC-containing Cannabis powders were prepared by mixing various ratios of THC distillate, commercially available from, e.g., https://www.thconcentrates.ca, and α-cyclodextrin. The THC distillate (300 mg) was diluted with isopropyl alcohol (4.2 mL) and then added to an α-cyclodextrin aqueous solution to generate suspensions having a ratio of α-cyclodextrin to THC distillate of 70:1, 87:1, 105:1 and 122:1. The resulting suspensions were shaken vigorously by hand in order to evenly distribute the diluted distillate throughout the suspensions. The suspensions were then rocked gently for 3 hours before being transferred to a drying dish. The suspensions were dried at ambient temperature for 48-72 hours or at 90-120° C. for 1-3 hours. Sufficient amounts of each resulting powder were added to water to produce solutions having a THC concentration of 10 mg/L. The solutions were then tasted by volunteers. The results are presented in Table 1. Table 1 recites the ratios of α-cyclodextrin (α-CD) to THC distillate and the taste characteristics of THC-containing solutions. All the solutions showed opalescence that decreased as the percent α-cyclodextrin increased and the ratio of α-cyclodextrin to distillate increased.

TABLE 1

| | α-CD: | α-CD:THC | |
|---|---|---|---|
| % α-CD | Isopropanol | Distillate | Solution Characteristics |
| 20 | 0.20 | 70:1 | Slight “off” taste, like stale |
| | | | water, with a thin and sparse |
| | | | layer on top of water |
| 25 | 0.16 | 87:1 | No taste, small film on surface |
| | | | of water |
| 30 | 0.13 | 105:1 | No taste, all solid material |
| | | | dissolved |
| 35 | 0.11 | 122:1 | No taste, all solid material |
| | | | dissolved |

The following Table 2 provides examples of Cannabis plant extract-derived compositions prepared from different Cannabis plant extracts, i.e., a tar, a distillate or an oil according to methods of the invention. “% Starting Material” in Table 2 refers to the approximate % mass (tar) or volume (distillate or oil) of the original tar, distillate, or oil mixed with an aqueous α-cyclodextrin composition. “% α-CD” is the % of α-cyclodextrin by weight in the aqueous cyclodextrin-containing composition mixed with the Cannabis plant extract. “End Product” means the Cannabis plant extract-derived composition produced by treating the plant extract with the aqueous α-cyclodextrin composition as described herein and dried to a powder form or not dried such that the end-product is in the form of a slurry.

TABLE 2

| | Staring Material | End Product | % α-CD | % Starting Material |
|---|---|---|---|---|
| 1 | Tar | Powder | 30 | 0.3 |
| 2 | Distillate | Powder | 25 | 0.3 |
| 3 | Commercial Oil | Slurry | 21 | 6.7 |

### Example 2—Tasteless, Odorless Cannabis Powder

A high CBD-containing Cannabis viscous oil was diluted 1:15 (w/v) with either ethyl or isopropyl alcohol and gently mixed using a tube rocking apparatus, for up to 3 hours to make the viscous oil less viscous. An α-cyclodextrin slurry was prepared by adding 900 mg of α-cyclodextrin to 10 mL of a 10% (w/v) aqueous α-cyclodextrin solution. 600 μL of the above-mentioned alcohol diluted Cannabis viscous oils was added to the aqueous α-cyclodextrin slurry to form a suspension. The suspension was shaken vigorously by hand for 30 seconds and then gently mixed at ambient temperature for at least 1 hour or until all visible signs of the deep green diluted viscous oil disappear. The suspension was shaken vigorously by hand for a few seconds and then poured into an evaporating dish. The suspension was dried at ambient temperatures for at least 48 hours or at 90-120° C. for 1-3 hours, scraped into a mortar, and ground to a fine greenish-tinged off-white powder. The powder was odorless, tasteless, and water soluble. Recovery based upon the quantity of solids in the initial mix of oil and α-cyclodextrin slurry was near 100%.

### Example 3—Tasteless, Odorless Cannabis Powder

A commercially-available cannabinoid oil [Tweed https://www.tweed.com] that had been diluted with vegetable oil by the supplier to contain 5.16 mg/mL THC and 4.77 mg/mL CBD was made into an off-white tasteless and odorless powder as follows. 600 mg of α-cyclodextrin was suspended in 7 mL of 10% (w/v) α-cyclodextrin in water. To this α-cyclodextrin suspension, 420 μL of the commercially available cannabinoid oil was added and briefly shaken by hand. The suspension was then mixed gently overnight or until all signs of the oil disappeared and the suspension appeared homogenous. The homogenous suspension was transferred to an evaporating dish and dried at ambient temperature for 48-72 hours. The dried material was transferred to a mortar and ground to an off-white waxy powder. At least a portion of this material was not soluble in cold water and formed small oil droplets in hot water. However, this material was tasteless and odorless. Analysis of the commercially available cannabinoid oil demonstrated that 2.0 mg of CBD and 2.17 mg of THC were added to the α-cyclodextrin suspension. Analysis of the off-white waxy powder generated by the method demonstrated 0.12% (w/w) THC and 0.13% (w/w) CBD. Recovery based upon total solids was near 100%.

### Example 4—Tasteless, Odorless Cannabis Suspension for Baking

3.0 mL of a commercially-available Cannabis oil (Spectrum Cannabis, https://spectrum Cannabis.com) containing 25 mg/mL THC was added to 45 mL of water containing 9.5 g α-cyclodextrin powder to make a suspension. The suspension was shaken vigorously by hand for one minute and then gently rocked for 3 hours. The resulting slurry was then added to a commercially available cupcake mix (e.g., Betty Crocker Vanilla Butter Cream) as a direct substitute for the same amount of water required by the manufacturer's cupcake recipe. As instructed by the cupcake recipe, the batter was divided into 12 equal portions and baked as indicated by the cupcake recipe. The olfactory and organoleptic properties of the resulting cupcakes were indiscernible from those of cupcakes prepared according to the unaltered cupcake recipe.

### Example 5—Tasteless, Odorless, High CBD-Containing Beverage

High CBD Cannabis containing viscous oil (50.9 mg CBD/100 mg oil) was diluted 1:15 (w/v) with isopropyl alcohol with gentle rocking until the oil was totally dissolved. The diluted oil (1.6 mL) was added to 10% (w/v) α-cyclodextrin aqueous solution (40 mL) containing additional solid α-cyclodextrin (8 g). The suspension was briefly mixed vigorously by hand before being rocked gently for 3-4 hours. The resulting homogeneous-appearing slurry was poured into a drying dish, and the solvents in the slurry were evaporated over several hours at approximately 110° C. Off-white crystalline material was recovered, and the yield (based on the measured amount of cannabinoid in the diluted oil) based on total mass of solids was 97%. The off-white crystalline material was transferred to a mortar and ground to a fine off-white powder. Analysis of the powder indicated that the powder contained 0.46% (w/w) CBD. The off-white powder (1.3 g of powder, the equivalent of 6 mg CBD) was dissolved in water (500 mL). The resulting solution was filtered through filter paper to remove traces of green material, believed to be vegetable matter. Although the resulting solution had a slight opalescence, the solution was transparent and tasteless. A volunteer suffering from chronic trigeminal nerve facial pain consumed approximately one-third of the CBD-containing solution. The volunteer reported pain relief within 10-15 minutes and reported that the relief lasted for over 4 hours after consuming the CBD-containing solution.

### Example 6—Tasteless, Odorless, High THC-Containing Iced Tea Beverage

The procedure used to make a high THC iced tea beverage was similar to the procedure used in Example 5. A THC-containing powder was prepared from a high THC-containing distillate as set forth in Example 4 and a sufficient amount of the powder was mixed with an iced tea beverage such that the resulting iced tea beverage contained 2.5 mg THC in 341 ml. Eight volunteers, two suffering from anxiety (group 1), three suffering from headaches (group 2) and three having neither headaches or anxiety (group 3), consumed 341 ml of the iced tea beverage and reported rapid onset of the desired effects, i.e., reduction of anxiety (group 1), reduction of headache (group 2) and an enhanced relaxation, happiness, and heightened sensory perception (groups 1, 2 and 3) that persisted for more than 2.5 hours.

None of the eight volunteers reported detecting any Cannabis and/or terpene taste or odor typically associated with Cannabis extract in the iced tea beverage. The volunteers reported that they only detected the flavor and odor of the tea in the iced tea beverage.

### Example 7—Preparing Tasteless, Odorless Cannabis Powder from a Cannabis Oil

High CBD containing Cannabis extract with the consistency of a tar was diluted 1:15 (w/v), with isopropyl alcohol and gently mixed by rocking the tube on a rocking apparatus for up to 3 hours to make the tar less viscous. An α-cyclodextrin slurry was prepared by adding 12g of α-cyclodextrin to 40 mL of water followed by vigorous shaking by hand for 2 minutes. 1.6 mL of the above-mentioned alcohol diluted Cannabis tar was added to the aqueous α-cyclodextrin slurry to form a suspension. This suspension was shaken vigorously by hand for 30 seconds and then gently mixed for at least 1 hour or until all visible signs of the deep green diluted tar disappear. The suspension was shaken vigorously by hand for a few seconds and then poured into an evaporating dish. The suspension was dried at ambient temperature for at least 48 hours, scraped into a mortar, and ground to a fine off-white powder. The yield, based upon solids added, was near 100%.

### Example 8—Tasteless, Odorless, Water-Soluble THC-Containing Cannabis Powder

A commercially available THC distillate (300 mg) (https://www.thconcentrates.ca) was diluted with isopropyl alcohol (4.2 mL). The diluted distillate (1.6 mL) was added to 40 mL of water containing 10 g of α-cyclodextrin. The resulting suspension was shaken vigorously by hand in order to evenly distribute the diluted distillate throughout the suspension. The suspension was then rocked gently for 3 hours before being transferred to a drying dish. The suspension was dried at ambient temperature for at least 48 hours to yield a powder containing 11.6 mg THC per 1 g powder.

### Example 9—Tasteless, Odorless, THC Water Beverage

The powder of Example 8(435 mg) was dissolved in 500 ml water. Brief mixing produced a tasteless, odorless THC-containing water beverage containing 5 mg THC.

### Example 10—Tasteless, Odorless, THC-Containing Tea Beverage

The powder of Example 8 (174 mg) was added to a teacup containing one tea bag of commercially available herbal tea (Stress Buster, www.tealish.com). The powder was dissolved completely upon addition of boiling water (250 mL). After steeping the tea bag for 5 minutes, an herbal tea beverage containing 2 mg THC was produced.

As described in the following examples, surprisingly, water-soluble, odorless, and tasteless cannabinoids were prepared without using water as a cosolvent.

### Example 11—Preparation of 10 mg THC Per Gram of Alpha-Cyclodextrin

Sixty-seven milligrams of (about 95%) THC Distillate was diluted in 500 μL of isopropyl alcohol. Five grams of α-cyclodextrin was added to a large-mouth jar with a nonreactive solid object to facilitate mixing. Four hundred eighty-five microliters of the diluted distillate was added to the α-cyclodextrin and the container was sealed. Mixing was conducted by inversion until the distillate was evenly dispersed in the α-cyclodextrin, for about 4-14 hours. Alcohol was removed by evaporation at 60° C. to produce a final product. The concentration of THC (cannabinoid) achieved in the final product was about 10 mg of cannabinoid per gram of cyclodextrin. One gram of the final product was dissolved in 500 mL of refrigerated bottled water (e.g., Walmart, Natural Spring Water). The resulting beverage was odorless, tasteless, slightly opalescent, very refreshing and contained 10 mg/500 mL THC.

### Example 12—Preparation of 20 mg THC Per Gram of Alpha-Cyclodextrin

THC Distillate (about 95%) at a weight of 133 mg was diluted in 500 μL of isopropyl alcohol. Five grams of α-cyclodextrin was added to a large-mouth jar with a nonreactive solid object to facilitate mixing. Four hundred eighty-five microliters of the diluted distillate was added to the α-cyclodextrin and the container was sealed. The container was mixed by inversion until the distillate was evenly dispersed in the α-cyclodextrin, for about 4-14 hours. The alcohol was removed by evaporation at 60° C. to produce a final product. The concentration of THC (cannabinoid) obtained in the final product was about 20 mg/g of cyclodextrin. One-half gram of the final product was dissolved in 500 mL of refrigerated bottled water (e.g., Walmart, Natural Spring Water). The resulting beverage was odorless, tasteless, slightly opalescent, very refreshing and contained 10 mg/500 mL THC.

### Example 13—Preparation of 50 mg THC Per Gram of Alpha-Cyclodextrin

Two hundred seventy milligrams of (about 95%) THC Distillate was dissolved in 2.0 mL of isopropyl alcohol. To a large-mouth jar, 5 g α-cyclodextrin was added, and a nonreactive solid object was also added to facilitate mixing. Then, 1.852 mL of the diluted distillate was added to the α-cyclodextrin, and the container was sealed. The contents of the container were mixed by inversion until the distillate was evenly dispersed in the α-cyclodextrin, for about 4-14 hours. The alcohol was removed by evaporation at 60° C. to produce a final product. The obtained concentration of THC (cannabinoid) in the final product was about 50 mg/g of cyclodextrin. Two hundred milligrams of the final product was dissolved in 500 mL of refrigerated bottled water (e.g., Walmart, Natural Spring Water). The resulting beverage was odorless, tasteless, slightly opalescent, very refreshing and contained 10 mg/500 mL THC.

### Example 14—Preparation of 20 mg CBD Per Gram of Alpha-Cyclodextrin

CBD Distillate (Veridesco, Full Spectrum CBD Distillate, 78% CBD) at a weight of 540 mg was diluted in 4.02 mL of ethyl alcohol. 3.7124, of the diluted distillate was added to 20 grams of α-cyclodextrin in a small blender (NutriBullet, www.nutribulet.com). The CBD/α-cyclodextrin mixture was blended for 10 seconds at which time the sides and the bottom of the blender were scraped down. This blending process was repeated 3-4 times until a very fine and homogeneous in appearance, damp powder was obtained. This damp powder was transferred to a drying dish and dried to a fine white powder at 60° C. for one hour. Surprisingly, a timed study demonstrated that the CBD/α-cyclodextrin complex formation was complete without further incubation or mixing.

### Example 15—Preparation of a CBD-Containing Citrus-Flavored Beverage

As described above in Example 14 prepare dry powder to contain 20 mg CBD per gram of α-CD. Add 500 mg of this powder (10 mg CBD) to a sufficient quantity of commercially available powdered citrus beverage (Crystal Light, Kraft Heinz, Canada) to prepare 500 mL of beverage. Dissolve the combined powders in 500 mL of cold water, and add ice to taste, if desired.

### Example 16—Preparation of a CBD-Containing Iced Tea Beverage

As described above in Example 14 prepare dry powder to contain 20 mg CBD per gram of α-CD. Add 500 mg of powder (10 mg CBD) to a sufficient quantity of commercially available powdered iced tea beverage (Nestea, Nestlé, Canada) to prepare 500 mL of beverage. Dissolve the combined powders in 500 mL of cold water. Add ice if desired.

### Example 17—Preparation of a CBD-Containing Sports Recovery Drink

As described above in Example 14 prepare dry powder to contain 20 mg CBD per gram of α-CD. Add 500 mg of powder (10 mg CBD) to a sufficient quantity of commercially available powdered citrus beverage (Crystal Light, Kraft Heinz, Canada) to prepare 500 mL of beverage. Add 800 mg of trisodium citrate and 104 mg of potassium chloride. Dissolve the combined powders in 500 mL of cold water. Add ice as desired.

### Example 18—Preparation of 15 mg CBD Per Gram of Alpha-Cyclodextrin Full Spectrum Powder

CBD Full Spectrum Organic CBD Distillate (Veridesco, Full Spectrum CBD Distillate, 58% CBD, 2.8% THC, 2.4% CannaBiChromene, 0.8% CannaBiGerol, 0.3% CannaBiDiVarin and 0.2% Other Cannabinoids) at a weight of 675 mg was diluted in 4.02 mL of ethyl alcohol. The diluted distillate, at a volume of 3.712μL, was added to 20 grams of α-cyclodextrin in a small blender (NutriBullet, www.nutribullet.com).

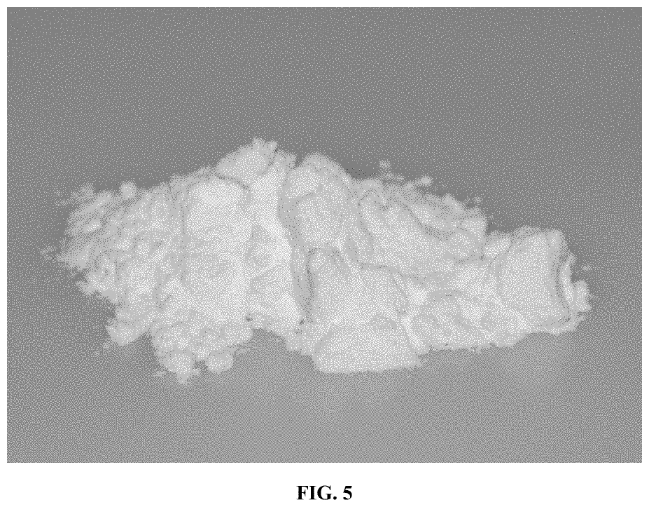

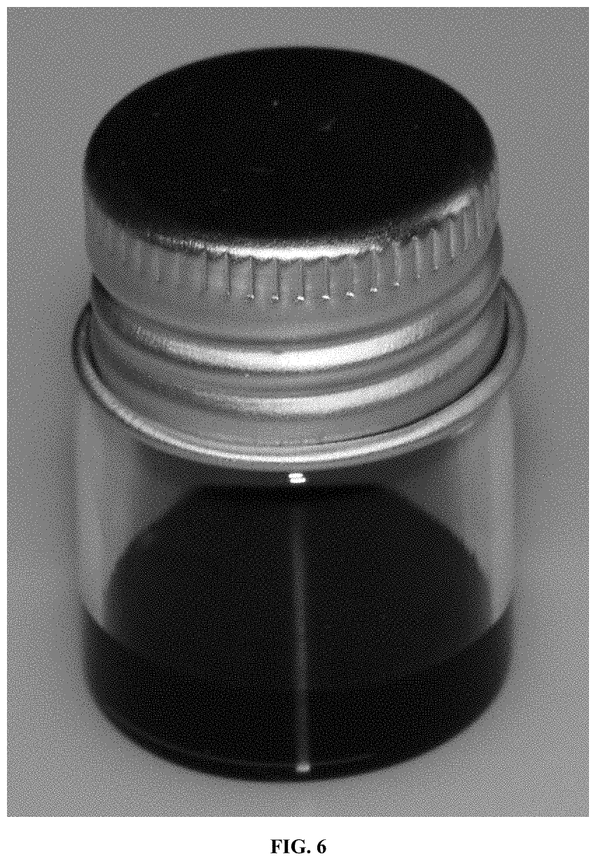

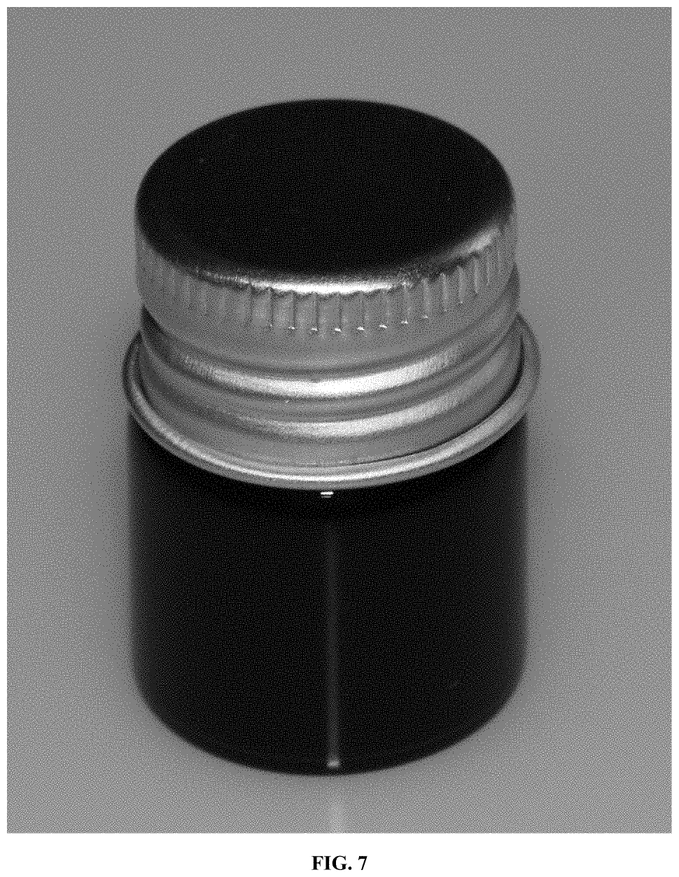

The alpha-cyclodextrin starting material that was used is a pure, white free-flowing powder as illustrated in FIG. 5. The undiluted full spectrum distillate (58% CBD, 2.8% THC, 2.4% CannaBiChromene, 0.8% CannaBiGerol, 0.3% CannaBiDiVarin and 0.2% Other Cannabinoids) that was used is very viscous and dark green, tending to black, in appearance as illustrated in FIG. 6. When the distillate was diluted with ethyl alcohol, the diluted distillate is less viscous (and thus easier to work with) but is still very dark in color as illustrated in FIG. 7.

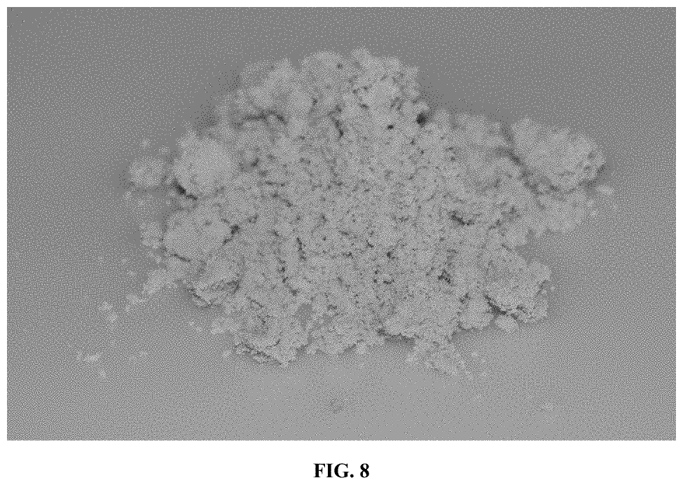

The cannabinoid/terpene/α-cyclodextrin mixture was blended for 10 seconds at which time the sides and the bottom of the blender were scraped down. This blending process was repeated 3-4 times until a very fine and homogeneous in appearance, damp powder was obtained. The obtained damp powder is illustrated in FIG. 8.

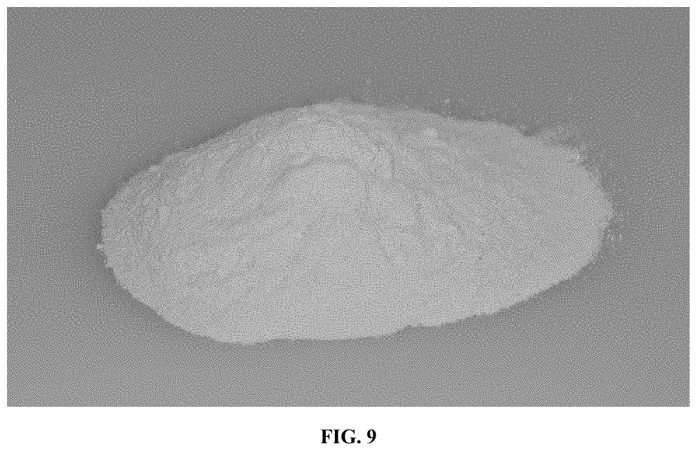

This obtained damp powder was transferred to a drying dish and dried to a fine light yellow powder at 60° C. for one hour. The dried cannabinoid-containing powder is illustrated in FIG. 9. This cannabinoid-containing powder after evaporation of ethyl alcohol is fine and free-flowing. As can be observed in FIG. 9, a slight yellowish tinge remains in the distillate. This yellowish color decreases to an off-white as the purity of the distillate increases.

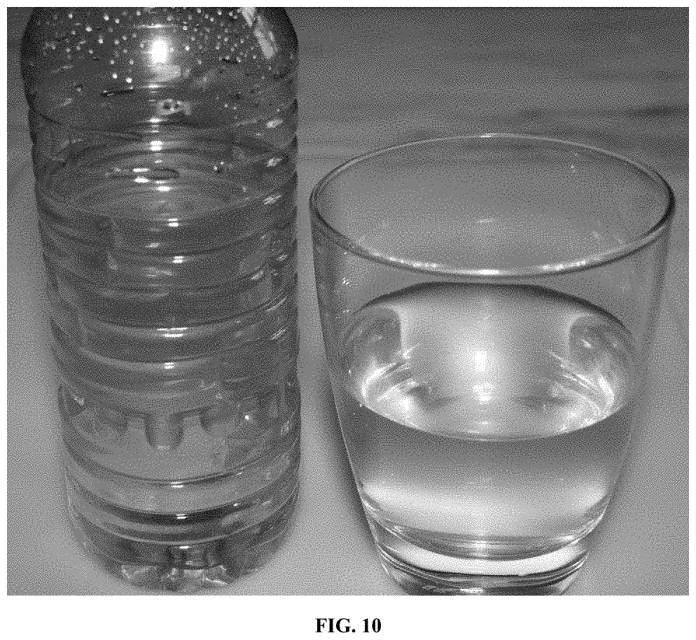

The dried cannabinoid-containing powder of FIG. 9 is dissolved in water at a concentration of 15 mg/L of CBD. The resulting solution can be seen in FIG. 10. As can be observed from FIG. 10, there is a slight yellowish tinge to the water as a result of impurities in the distillate; nonetheless, the water is odorless and tasteless with a slight opalescence.

### Example 19—Improved Absorption of Cannabinoid Complexed with α-CD

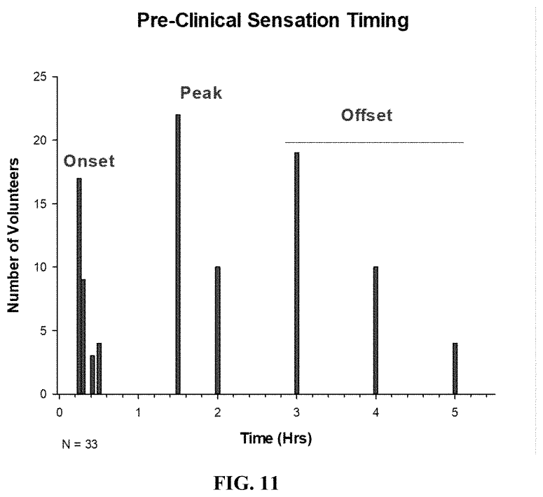

Hydrophobic molecules tend to be absorbed very poorly. Cannabinoids that are complexed by α-CD are water soluble and are absorbed (beginning in the mouth) much more efficiently than cannabinoids not complexed by α-CD. As a result, substantially less α-CD-complexed cannabinoid needs to be consumed in order to elicit similar effects to products where cannabinoid is not complexed with α-CD. As evident from FIG. 11, the onset of effects took between 10 and 30 minutes, which is consistent with oral (as opposed to intestinal) absorption. Peak absorption is in the range of 90-120 minutes and is consistent with additional intestinal absorption. It is also of note that the desired effects of the α-CD complexed cannabinoid are experienced for a duration of four to five hours.

The foregoing description and examples have been set forth merely to illustrate the invention and are not meant to be limiting. Since modifications of the described embodiments incorporating the spirit and the substance of the invention may occur to persons skilled in the art, the invention should be construed broadly to include all variations within the scope of the claims and equivalents thereof.

1. A method for making a composition comprising complexes of α-cyclodextrin and one or more cannabinoids, one or more terpenes, or mixtures of such complexes, the method comprising the steps of:
(a) combining a first composition comprising at least one cannabinoid and/or at least one terpene or mixtures of such cannabinoids and terpenes with a non-aqueous α-cyclodextrin-containing composition; and
(b) mixing the first composition and the non-aqueous α-cyclodextrin-containing composition for a sufficient time to form complexes between the α-cyclodextrin and at least one cannabinoid and/or at least one terpene.

2. The method of claim 1, wherein the first composition is a non-aqueous solid.

3. The method of claim 1, wherein the first composition is dissolved in ethyl or isopropyl alcohol.

4. The method of claim 1, further comprising the step of:
(c) removing diluents from step (b).

5. The method of claim 1, wherein the first composition is a Cannabis plant extract comprising one or more phytocannabinoids or a composition comprising one or more synthetically-produced cannabinoids.

6. The method of claim 5, wherein the phytocannabinoid is THC, CBD, cannabinol, or mixtures thereof and wherein the synthetic cannabinoid is 1-pentyl-3-(1-naphthoyl)indole (JWH-018), 1-butyl-3-(1-naphthoyl)indole (JWH-073), 1-[2-(4-morpholinyl)ethyl]-3-(1-naphthoyl)indole (JWH-200), 5-(1,1-dimethylheptyl)-2-[(1R,3S)-3-hydroxycyclohexyl]-phenol (CP-47,497), and 5-(1,1-dimethyloctyl)-2-[(1R,3S)-3-hydroxycyclohexyl]-phenol (cannabicyclohexanol; CP-47,497 C8 homologue) dronabinol, nabilone, or mixtures thereof.

7. The method of claim 1, wherein the non-aqueous α-cyclodextrin-containing composition is a powder, crystals, or granules.

8. The method of claim 1, wherein the non-aqueous α-cyclodextrin comprises about 1000:1 to about 2:1 w/w of α-cyclodextrin to the first composition.

9. The method of claim 4, wherein the mixture is dried by evaporating the mixture in dishes or pans at ambient or moderately elevated temperatures, at atmospheric or reduced pressure, or in a rotary evaporator.

10. A method for complexing a compound in a Cannabis plant extract with a non-aqueous α-cyclodextrin comprising the steps of:
(a) combining the Cannabis plant extract with the non-aqueous α-cyclodextrin to produce a mixture,
(b) mixing the mixture from step (a) for sufficient time to form a composition comprising complexes of α-cyclodextrin with the compound in the Cannabis plant extract,
wherein the compound is a cannabinoid, a terpene, and/or mixtures thereof.

11. The method of claim 10, wherein the compound in the Cannabis plant extract is a cannabinoid selected from the group consisting of cannabinol, THC, and CBD.

12. A food product comprising a composition comprising complexes of one or more cannabinoids and α-cyclodextrin produced by the method of claim 1.

13. The food product of claim 12, wherein the food product is a farinaceous food product, a non-farinaceous food product, or a confection.

14. The food product of claim 13, wherein the food product is a farinaceous food product selected from the group consisting of cake, muffin, brownie, cookie, cracker, doughnut, and biscuit or,
the food product is a confection selected from the group consisting of caramel, chocolate, nougat, lozenge, chewing gum, toffee, fondant, halvah, jelly, gelatin, gummies, suckers, lollipops, licorice, marshmallow, taffy, or marzipan, or
the food product is a beverage selected from the group consisting of a water, a tea, a coffee, a juice, a carbonated or uncarbonated beverage, an alcoholic beverage, a mocktail, a sports drink, or a smoothie.

15. A topical formulation comprising a composition comprising complexes of α-cyclodextrin and cannabinoids, complexes of α-cyclodextrin and terpenes, or mixtures of such produced by the method of claim 1.

16. The topical formulation of claim 15, wherein the topical formulation is a lotion, a cream, or an oil.

17. A composition comprising complexes of a non-aqueous α-cyclodextrin and a phytocannabinoid, complexes of non-aqueous α-cyclodextrin and a synthetically produced cannabinoid, complexes of non-aqueous α-cyclodextrin and terpenes, or mixtures of two or more of such complexes, formulated such that the complexes are water soluble.

18. A food product comprising the composition of claim 17.

19. A method of treating a subject having a disorder, comprising administering a therapeutically effective amount of the composition of claim 17 to the subject having the disorder.

20. The method of claim 19, wherein the disorder is agitation, Alzheimer's dementia, anxiety disorders, jet lag, depression, anorexia, Attention Deficit Hyperactivity Disorder (ADHD) with or without hyperactivity, Autism Spectrum Disorder (ASD), cachexia, cancer, chronic cluster headaches, Crohn's disease, convulsions, epilepsy, gastrointestinal tract distress (i.e., heartburn indigestion, stomach ache, etc.), glaucoma, HIV/AIDS, Huntington's disease, inflammation (chronic or acute), inflammatory bowel disease (IBS), irritable bowel syndrome (IBS) with or without diarrhea or constipation, migraine headaches whether hormone mediated or not, muscle spasms, nausea, neurodegenerative diseases including Lou Gehrig's disease, all forms of pain (i.e., acute chronic, neuropathic, trigeminal nerve pain, and pain associated with or accompanying migraine headaches and cancer), Parkinson's disease and Parkinsonian-type symptoms, spinal-cord injuries, postmenstrual syndrome (PMS), Post-Traumatic Stress Disorder (PTSD), seasonal affective disorder (SAD), seizures, seizures associated with Lennox-Gastaut syndrome or Dravet syndrome, sleep disorders such as insomnia, spasticity from multiple sclerosis, ulcerative colitis (UC), or vomiting.
